(19) United States
(12) Patent Application Publication
(10) Pub. No.: US 2025/0033051 A1
(43) Pub. Date: Jan. 30, 2025

(54) BIOPARTICLE ENRICHMENT APPARATUS, BIOPARTICLE ENRICHMENT DEVICE, AND PICO-DROPLET GENERATOR

(71) Applicant: CYTOAURORA BIOTECHNOLOGIES, INC., HSINCHU COUNTY (TW)

(72) Inventors: Chung-Er Huang, HSINCHU COUNTY (TW); Hsin-Cheng Ho, HSINCHU COUNTY (TW)

(21) Appl. No.: 18/507,075

(22) Filed: Nov. 12, 2023

(30) Foreign Application Priority Data
Jul. 27, 2023 (TW) 112128063

(51) Int. Cl. B01L 3/00; G01N 15/10

(52) U.S. Cl. CPC B01L 3/502761; G01N 15/10; B01L 2200/027; B01L 2200/0647; B01L 2300/0645; B01L 2300/0654; B01L 2300/087; B01L 2300/16; B01L 2400/027; B01L 2400/0424; G01N 2015/0019; G01N 15/01

(57) ABSTRACT

A pico-droplet generator includes a light sensing structure, a mating structure that is spaced apart from the light sensing structure, a bonding layer that connects the light sensing structure and the mating structure, and a piezoelectric member that is disposed on the bonding layer. The pico-droplet generator defines a selection channel along a flowing direction. The bonding layer has a selection hole that is in spatial communication with the selection channel along a dripping direction perpendicular to the flowing direction. The piezoelectric member and the selection hole are respectively arranged on two opposite sides of the bonding layer. The pico-droplet generator has a pico-droplet emission region that is defined from the selection hole toward the piezoelectric member.

### CROSS-REFERENCE TO RELATED PATENT APPLICATION

This application claims the benefit of priority to Taiwan Patent Application No. 112128063, filed on Jul. 27, 2023. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a droplet generator, and more particularly to a bioparticle enrichment apparatus, a bioparticle enrichment device, and a pico-droplet generator.

### BACKGROUND OF THE DISCLOSURE

An enrichment process is the first step in conventional research for biological particles, but the conventional enrichment process still requires a significant amount of manpower. Accordingly, how apparatuses or devices can be incorporated in the process to quickly and accurately implement the enrichment process has been one of the important areas of research and development in the relevant field.

### SUMMARY OF THE DISCLOSURE

In response to the above-referenced technical inadequacies, the present disclosure provides a bioparticle enrichment apparatus, a bioparticle enrichment device, and a pico-droplet generator for effectively improving on the issues associated with conventional enrichment processes.

In order to solve the above-mentioned problems, one of the technical aspects adopted by the present disclosure is to provide a bioparticle enrichment apparatus provided for selecting at least one of bioparticles from a liquid specimen having the bioparticles. The bioparticle enrichment apparatus includes a pico-droplet generator, a power device, a pressure balance device, a camera device, and a control device. The pico-droplet generator includes a light sensing structure, a mating structure, a bonding layer, and a piezoelectric member. The light sensing structure includes a first substrate, a first electrode layer and a photoelectric layer respectively formed on two opposite sides of the first substrate, and an insulating layer covering the photoelectric layer. The mating structure is spaced apart from the light sensing structure. At least one of the mating structure and the light sensing structure is transparent, and the mating structure includes a second substrate that faces toward the light sensing structure and a second electrode layer that is formed on the second substrate. The bonding layer is connected in-between the light sensing structure and the mating structure along a thickness direction so as to jointly define a selection channel along a flowing direction perpendicular to the thickness direction. At least one of the mating structure and the bonding layer has an inlet that is located at an upstream of the selection channel and an outlet that is located at a downstream of the selection channel. The bonding layer has a selection hole that is in spatial communication with the selection channel along a dripping direction perpendicular to the thickness direction and the flowing direction. The piezoelectric member is disposed on the bonding layer. The piezoelectric member and the selection hole are respectively located at two opposite sides of the bonding layer along the dripping direction. The pico-droplet generator has a pico-droplet emission region defined as extending from the selection hole toward the piezoelectric member. The power device is electrically coupled to the first electrode layer and the second electrode layer. The pressure balance device includes a first valve that is assembled to the inlet and a second valve that is assembled to the outlet. The pressure balance device is configured to control a velocity and a pressure of the liquid specimen in the selection channel through the first valve and the second valve. The camera device corresponds in position to a viewable segment of the selection channel. The pico-droplet emission region is arranged in the viewable segment, and the camera device is configured to take a real-time image of the liquid specimen in the viewable segment. The control device is electrically coupled to the piezoelectric member. When the real-time image obtained by the camera device shows that at least one of the bioparticles is located in the pico-droplet emission region, the control device allows the piezoelectric member to output a pico-droplet by driving the liquid specimen in the pico-droplet emission region to pass through the selection hole, and the pico-droplet covers the at least one of the bioparticles.

In order to solve the above-mentioned problems, another one of the technical aspects adopted by the present disclosure is to provide a bioparticle enrichment device provided for selecting a target bioparticle from a liquid specimen having bioparticles. The bioparticle enrichment device includes a pico-droplet generator and a light source mechanism. The pico-droplet generator includes a light sensing structure, a mating structure, a bonding layer, and a piezoelectric member. The light sensing structure includes a first substrate, a first electrode layer and a photoelectric layer respectively formed on two opposite sides of the first substrate, and an insulating layer covering the photoelectric layer. The mating structure is spaced apart from the light sensing structure. At least one of the mating structure and the light sensing structure is transparent, and the mating structure includes a second substrate that faces toward the light sensing structure and a second electrode layer that is formed on the second substrate. The bonding layer is connected in-between the light sensing structure and the mating structure along a thickness direction so as to jointly define a selection channel along a flowing direction perpendicular to the thickness direction. At least one of the mating structure and the bonding layer has an inlet that is located at an upstream of the selection channel and an outlet that is located at a downstream of the selection channel. The bonding layer has a selection hole that is in spatial communication with the selection channel along a dripping direction perpendicular to the thickness direction and the flowing direction. The piezoelectric member is disposed on the bonding layer. The piezoelectric member and the selection hole are respectively located at two opposite sides of the bonding layer along the dripping direction. The pico-droplet generator has a pico-droplet emission region defined as extending from the selection hole toward the piezoelectric member. The light source mechanism is configured to move the target bioparticle into the pico-droplet emission region by emitting light onto the bioparticle to apply a dielectrophoretic (DEP) force to the target bioparticle through the light sensing structure. The piezoelectric member is configured to output a pico-droplet by driving the liquid specimen in the pico-droplet emission region to pass through the selection hole, and the pico-droplet covers the target bioparticle.

In order to solve the above-mentioned problems, yet another one of the technical aspects adopted by the present disclosure is to provide a pico-droplet generator. The pico-droplet generator includes a light sensing structure, a mating structure, a bonding layer, and a piezoelectric member. The light sensing structure includes a first substrate, a first electrode layer and a photoelectric layer respectively formed on two opposite sides of the first substrate, and an insulating layer covering the photoelectric layer. The mating structure is spaced apart from the light sensing structure. At least one of the mating structure and the light sensing structure is transparent, and the mating structure includes a second substrate that faces toward the light sensing structure and a second electrode layer that is formed on the second substrate. The bonding layer is connected in-between the light sensing structure and the mating structure along a thickness direction so as to jointly define a selection channel along a flowing direction perpendicular to the thickness direction. At least one of the mating structure and the bonding layer has an inlet that is located at an upstream of the selection channel and an outlet that is located at a downstream of the selection channel. The bonding layer has a selection hole that is in spatial communication with the selection channel along a dripping direction perpendicular to the thickness direction and the flowing direction. The piezoelectric member is disposed on the bonding layer. The piezoelectric member and the selection hole are respectively located at two opposite sides of the bonding layer along the dripping direction. The pico-droplet generator has a pico-droplet emission region defined as extending from the selection hole toward the piezoelectric member.

Therefore, the configuration of the pico-droplet generator of the bioparticle enrichment apparatus provided by the present disclosure can be used to quickly and accurately implement a selection and enrichment process of the bioparticle (or the at least one target bioparticle) through the piezoelectric member. The pico-droplet generator in the present disclosure has an architecture that allows for mass-production of the same, thereby facilitating reduction of the overall cost of the bioparticle enrichment apparatus.

Moreover, the pico-droplet generator and the light source mechanism of the bioparticle enrichment apparatus provided by the present disclosure can be used in cooperation with each other to allow the pico-droplet emission region to quickly and accurately have the at least one target bioparticle therein, such that the at least one target bioparticle can be collected in the first container by forming the pico-droplet.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic planar view of a bioparticle enrichment apparatus according to a first embodiment of the present disclosure;

FIG. 2 is a schematic perspective view showing a part of FIG. 1;

FIG. 3 is a schematic cross-sectional view taken along line III-III of FIG. 2;

FIG. 4 is a schematic cross-sectional view showing a second valve of FIG. 3 being switched to a closed mode;

FIG. 5 is a schematic cross-sectional view showing a dielectrophoretic force in FIG. 4 applied to move bioparticles;

FIG. 6 is a schematic cross-sectional view showing a pico-droplet generator of FIG. 5 that outputs a pico-droplet through a piezoelectric member;

FIG. 7 is a schematic cross-sectional view showing the second valve of FIG. 6 being switched to an open mode;

FIG. 8 is a schematic cross-sectional view showing the bioparticle enrichment apparatus in another operation process according to the first embodiment of the present disclosure;

FIG. 9 is a schematic cross-sectional view showing the pico-droplet generator of FIG. 8 that outputs a pico-droplet through the piezoelectric member;

FIG. 10 is a schematic perspective view showing a part of the bioparticle enrichment apparatus according to a second embodiment of the present disclosure;

FIG. 11 is a schematic cross-sectional view taken along line XI-XI of FIG. 10;

FIG. 12 is a schematic enlarged view of part XII of FIG. 11; and

FIG. 13 is a schematic top view of a vertical transistor of FIG. 12.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

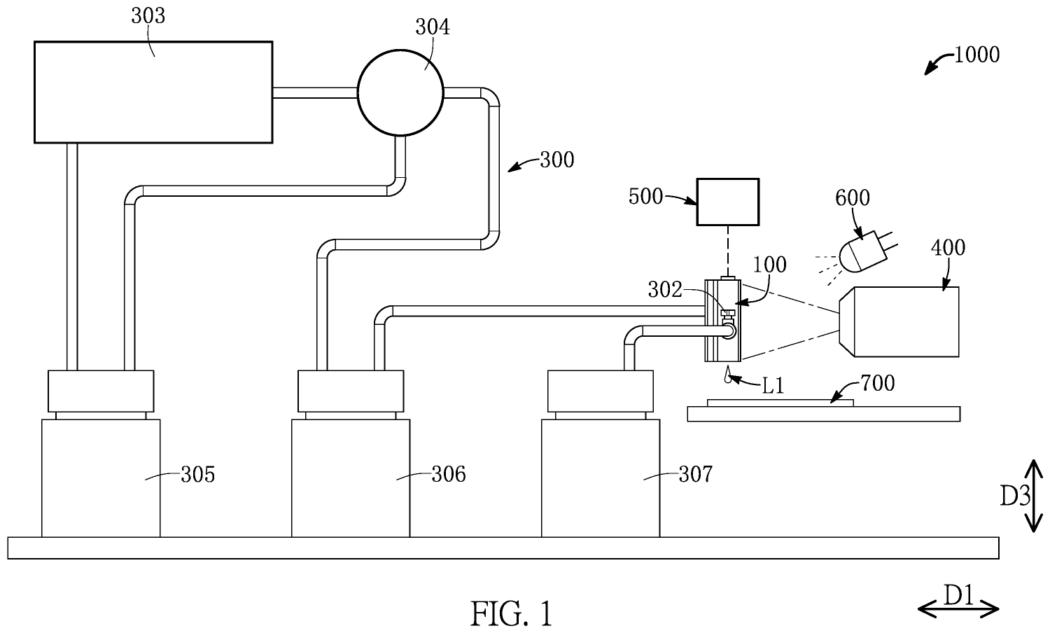

Referring to FIG. 1 to FIG. 9, a first embodiment of the present disclosure is provided. As shown in FIG. 1 to FIG. 3, the present embodiment provides a bioparticle enrichment apparatus 1000 for selecting at least one of bioparticles B from a liquid specimen L having the bioparticles B, thereby enabling the at least one of the bioparticles B to be collected in a first container 700.

In the present embodiment, the liquid specimen L can be a body fluid from an animal (e.g., blood, lymph, saliva, ascites, or urine), and the bioparticle B can be a specific type of cell, such as circulating tumor cells (CTCs), fetal nucleated red blood cells (FNRBCs), exosomes, virus, or bacteria, but the present disclosure is not limited thereto. For example, in other embodiments of the present disclosure not shown in the drawings, the liquid specimen L can also be obtained from plants.

Moreover, the bioparticle enrichment apparatus 1000 in the present embodiment includes a pico-droplet generator 100, a power device 200 electrically coupled to the pico-droplet generator 100, a pressure balance device 300 being in spatial communication with an interior space of the pico-droplet generator 100, a camera device 400 provided for observing the pico-droplet generator 100, a control device 500 provided for driving (or triggering) the pico-droplet generator 100, and a light source mechanism 600 that is used in cooperation with the pico-droplet generator 100 in a contactless manner, but the present disclosure is not limited thereto.

For example, in other embodiments of the present disclosure not shown in the drawings, configurations and number of inner components of the bioparticle enrichment apparatus 1000 can be changed or adjusted according to design requirements (e.g., the light source mechanism 600 can be omitted), and the pico-droplet generator 100 can be independently used (e.g., sold) or can be used in cooperation with other devices (e.g., the pico-droplet generator 100 and the light source mechanism 600 can be used in cooperation with each other and are jointly defined as a bioparticle enrichment device).

The following description describes the structure and connection relationship of each component of the bioparticle enrichment apparatus 1000, and then describes the connection relationship of each component of the bioparticle enrichment apparatus 1000. It should be noted that the pico-droplet generator 100 of the present embodiment is formed at a chip-scale (e.g., a thickness of the pico-droplet generator 100 is within a range from 600 μm to 800 μm), and the pico-droplet generator 100 shown in the drawings is a rectangular structure, but the present disclosure is not limited thereto. For example, in other embodiments of the present disclosure not shown in the drawings, the pico-droplet generator 100 can be a curved structure or an irregular structure.

Specifically, the pico-droplet generator 100 in the present embodiment includes a light sensing structure 1, a mating structure 2 that is spaced apart from the light sensing structure 1, a bonding layer 3 that is connected to and located between the light sensing structure 1 and the mating structure 2 along a thickness direction D1, and a piezoelectric member 4 that is disposed on the bonding layer 3. In order to clearly describe the pico-droplet generator 100, the mating structure 2 in the present embodiment is transparent. According to practical requirements, at least one of the mating structure 2 and the light sensing structure 1 can be transparent so as to enable the pico-droplet generator 100 to be normally operated.

The light sensing structure 1 includes a first substrate 11, a first electrode layer 12 formed on one side (e.g., a bottom side) of the first substrate 11, a photoelectric layer 13 formed on another side (e.g., a top side) of the first substrate 11, and an insulating layer 14 that covers at least part of the photoelectric layer 13. In the present embodiment, the first substrate 11 can be a silicon substrate and is preferably a low-doped N-type layer, the first electrode layer 12 can cover an entirety of the bottom side of the first substrate 11, and the first electrode layer 12 is preferably a thin conductive metal layer or an indium tin oxide (ITO) layer. Moreover, configuration or layout of the photoelectric layer 13 can be adjusted or changed according to design requirements, and the present disclosure is not limited thereto. For example, the photoelectric layer 13 can have a plurality of vertical transistors (now labeled in the drawings) in a matrix arrangement, and further details of the vertical transistors of the photoelectric layer 13 are described in the following second embodiment for the sake of brevity.

The mating structure 2 includes a second substrate 21 and a second electrode layer 22 that is formed on the second substrate 21 and that faces toward the light sensing structure 1. The bonding layer 3 is connected to and arranged between the light sensing structure 1 and the mating structure 2 so as to jointly define a selection channel 5 along a flowing direction D2 that is perpendicular to the thickness direction D1.

Specifically, at least one of the mating structure 2 and the bonding layer 3 has an inlet 31 and an outlet 32, which are respectively in spatial communication with two ends of the selection channel 5. In other words, the inlet 31 is located at an upstream of the selection channel 5, and the outlet 32 is located at a downstream of the selection channel 5. Moreover, the inlet 31 and the outlet 32 in the present embodiment are formed on the bonding layer 3, and the liquid specimen L can be injected into the pico-droplet generator 100 through the inlet 31 and can flow out of the pico-droplet generator 100 through the outlet 32.

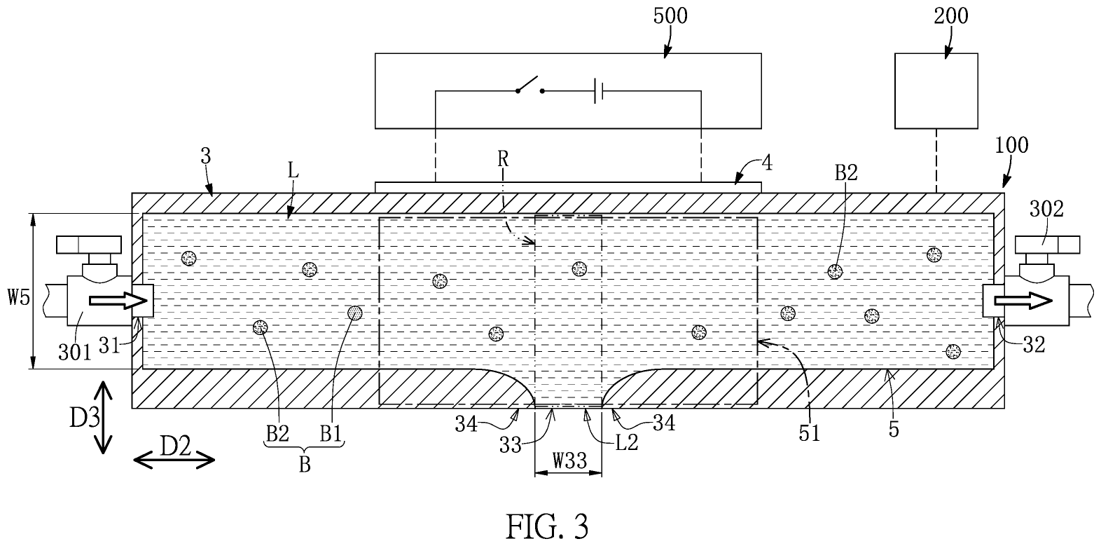

Furthermore, the bonding layer 3 has a selection hole 33 that is in spatial communication with the selection channel 5 along a dripping direction D3 perpendicular to the thickness direction D1 and the flowing direction D2 (e.g., the selection hole 33 penetrates through the bonding layer 3 along the dripping direction D3). In the present embodiment, the pico-droplet generator 100 preferably has a hydrophobic surface 34 surrounding the selection hole 33 (i.e., the selection hole 33 is surrounded and defined by the hydrophobic surface 34). The hydrophobic surface 34 in the present embodiment includes bottom edges of the bonding layer 3 (as shown in FIG. 3) respectively located at two opposite sides of the selection hole 33 along the flowing direction D2, a partial outer surface of the light sensing structure 1, and a partial outer surface of the mating structure 2, the latter two of which are respectively located at two opposite side of the selection hole 3 along the thickness direction D1 (not shown in the drawings). Moreover, the selection hole 33 is substantially arranged on a center portion of the bonding layer 3 along the flowing direction D2, a width W33 of the selection hole 33 along the flowing direction D2 is within a range from 40 μm to 300 μm, and the dripping direction D3 is substantially perpendicular to a horizontal plane or substantially parallel to a vertical line, but the present disclosure is not limited thereto.

The piezoelectric member 4 is disposed on a top edge of the bonding layer 3 and is located outside of the selection channel 5, and the piezoelectric member 4 and the selection hole 33 are respectively located at two opposite sides of the bonding layer 3 along the dripping direction D3. The pico-droplet generator 100 (or the selection channel 5) has a pico-droplet emission region R defined as extending from the selection hole 33 toward the piezoelectric member 4.

It should be noted that the pico-droplet emission region R in the present embodiment is arranged in a projection area defined by orthogonally projecting the selection hole 33 onto the piezoelectric member 4 along the dripping direction D3, and a boundary of the pico-droplet emission region R is spaced apart from a center of the selection hole 33 along the dripping direction D3 by a distance within a range from 50 μm to 200 μm. In other words, the pico-droplet emission region R can cover an entirety of a width W5 of the selection channel 5 along the dripping direction D3, and a volume of the pico-droplet emission region R can be substantially equal to that of at least one pico-droplet L1 according to design requirements.

In addition, the boundary of the pico-droplet emission region R can be changed or adjusted according to design requirements and is not limited by the drawings. For example, in other embodiments of the present disclosure not shown in the drawings, the pico-droplet emission region R can have the shape of a circular-sector defined as extending from the selection hole 3; or, the pico-droplet emission region R covers a part (e.g., 30% to 80%) of the width W5 of the selection channel 5 along the dripping direction D3.

The power device 200 in the present embodiment is an alternating current (AC) power device. The power device 200 is electrically coupled to the first electrode layer 12 and the second electrode layer 22 of the pico-droplet generator 100, thereby providing electricity for a cooperative operation of the pico-droplet generator 100 and the light source mechanism 600.

The pressure balance device 300 includes a first valve 301 that is assembled to the inlet 31 and a second valve 302 that is assembled to the outlet 32. The pressure balance device 300 is configured to control a velocity and a pressure of the liquid specimen L in the selection channel 5 through the first valve 301 and the second valve 302. For example, the pressure balance device 300 enables the liquid specimen L to form a liquid level L2 in the selection hole 33 that is coplanar with an outer surface (e.g., the bottom edges) of the bonding layer 3.

Specifically, the pressure balance device 300 in the present embodiment includes an air pump 303, a switch 304 connected to the air pump 303, a pressure balance bottle 305 being in fluid communication with the air pump 303 and the switch 304, a liquid injection bottle 306 being in fluid communication with the switch 304 and the first valve 301, and a second container 307 that is in fluid communication with the second valve 302. The liquid injection bottle 306 enables the liquid specimen L received therein to be injected into the selection channel 5 through the first valve 301 and the inlet 31. The second container 307 is provided to receive the liquid specimen L that is selected (or filtered) and flows out of the selection channel 5 from the outlet 32 and the second valve 302.

The camera device 400 corresponds in position to a viewable segment 51 of the selection channel 5, and the camera device 400 is preferably configured to take a real-time image of the liquid specimen L in the viewable segment 51, thereby determining a preferable operation time of the pico-droplet generator 100. It should be noted that the bioparticles B located in the viewable segment 51 can be divided into at least one target bioparticle B1 and at least one non-target bioparticle B2. Moreover, the camera device 400 faces toward the light sensing structure 1 being transparent or the mating structure 2 being transparent along thickness direction D1, thereby clearly observing a real-condition of the liquid specimen L that flows in the selection channel 5 along the flowing direction D2.

Specifically, a boundary of the viewable segment 51 is spaced apart from a center of the selection hole 33 along the flowing direction D2 by a distance within a range from 100 μm to 300 μm, the viewable segment 51 covers an entirety of the width W5 of the selection channel 5 along the dripping direction D3, and the pico-droplet emission region R is arranged in the viewable segment 51. In other words, the viewable segment 51 can be defined as a filtering segment, a portion of the selection channel 5 arranged at an upstream of the viewable segment 51 can be defined as an unfiltered segment, and another portion of the selection channel 5 arranged at a downstream of the viewable segment 51 can be defined as a filtered segment, but the present disclosure is not limited thereto.

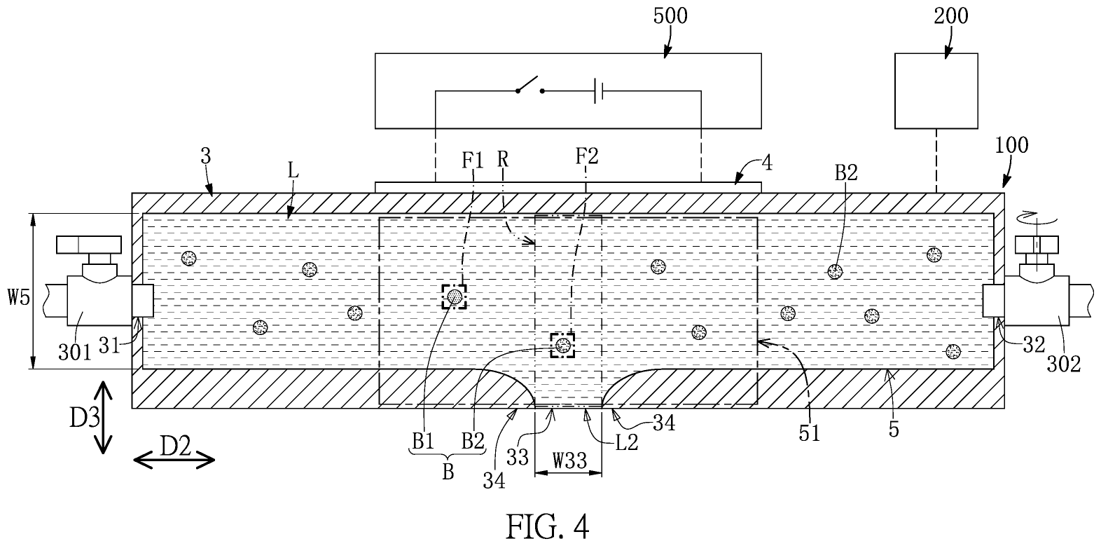

The control device 500 is electrically coupled to the piezoelectric member 4 of the pico-droplet generator 100, so that the control device 500 can be used in cooperation with the real-time image obtained by the camera device 400 to trigger (or drive) the piezoelectric member 4. It should be noted that when the piezoelectric member 4 (or the light source mechanism 600) is in operation, the pressure balance device 300 maintains the pressure of the liquid specimen L in the selection channel 5 through the first valve 301 in an open mode and the second valve 302 in a closed mode (as shown in FIG. 4 to FIG. 6).

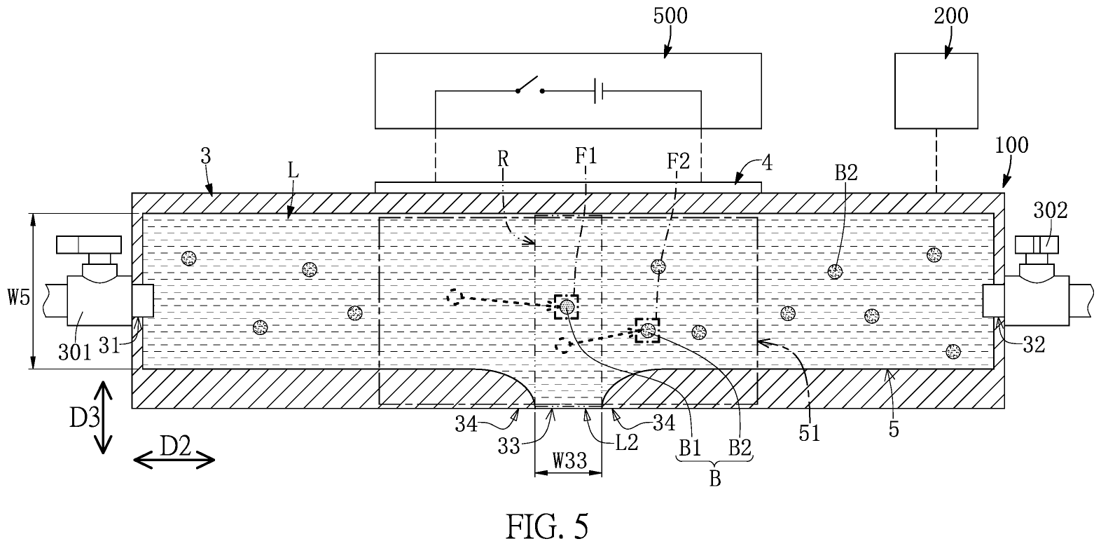

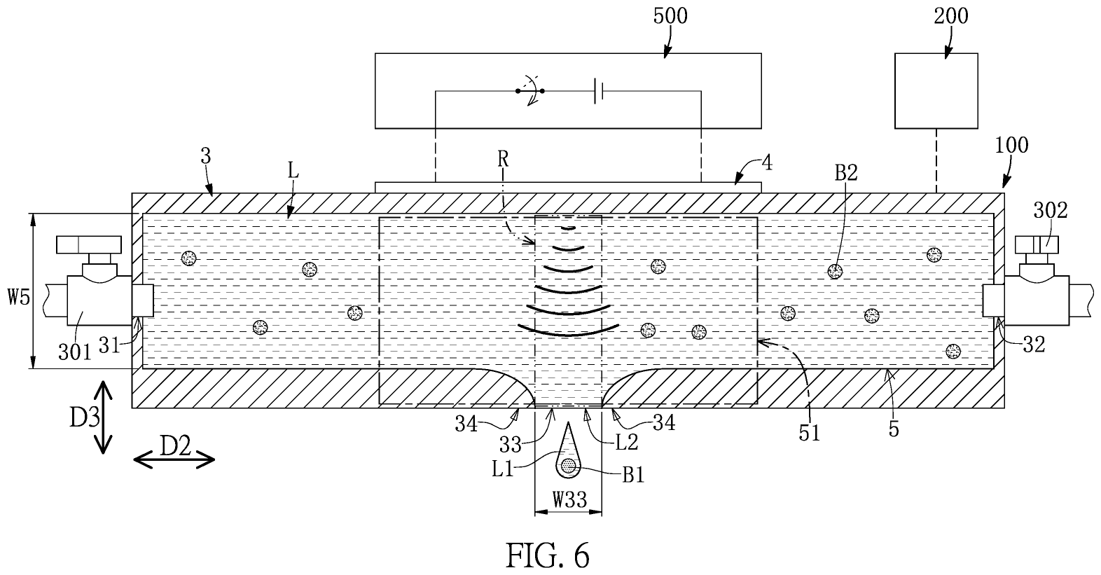

As shown in FIG. 1, FIG. 5, and FIG. 6, when the real-time image obtained by the camera device 400 shows that at least one of the bioparticles B (e.g., the at least one target bioparticle B1) is located in the pico-droplet emission region R, the control device 500 allows the piezoelectric member 4 to output one of the pico-droplets L1 by driving the liquid specimen L in the pico-droplet emission region R to pass through the selection hole 33. Moreover, the pico-droplet L1 covers the at least one of the bioparticles B (e.g., the at least one target bioparticle B1) and is received (or collected) in the first container 700.

In addition, if the bioparticle B (e.g., the at least one target bioparticle B1) is located away from the selection hole 33, the control device 500 enables the piezoelectric member 4 to sequentially output more than one of the pico-droplets L1 for ensuring that at least one of the pico-droplets L1 outputted from the pico-droplet generator 100 covers the bioparticle B (e.g., the at least one target bioparticle B1).

Accordingly, the configuration of the pico-droplet generator 100 of the bioparticle enrichment apparatus 1000 provided by the present embodiment can be used to quickly and accurately implement a selection and enrichment process of the bioparticle B (e.g., the at least one target bioparticle B1) through the piezoelectric member 4. The pico-droplet generator 100 in the present embodiment has an architecture that allows for mass-production of the same, thereby facilitating reduction of overall cost of the bioparticle enrichment apparatus 1000.

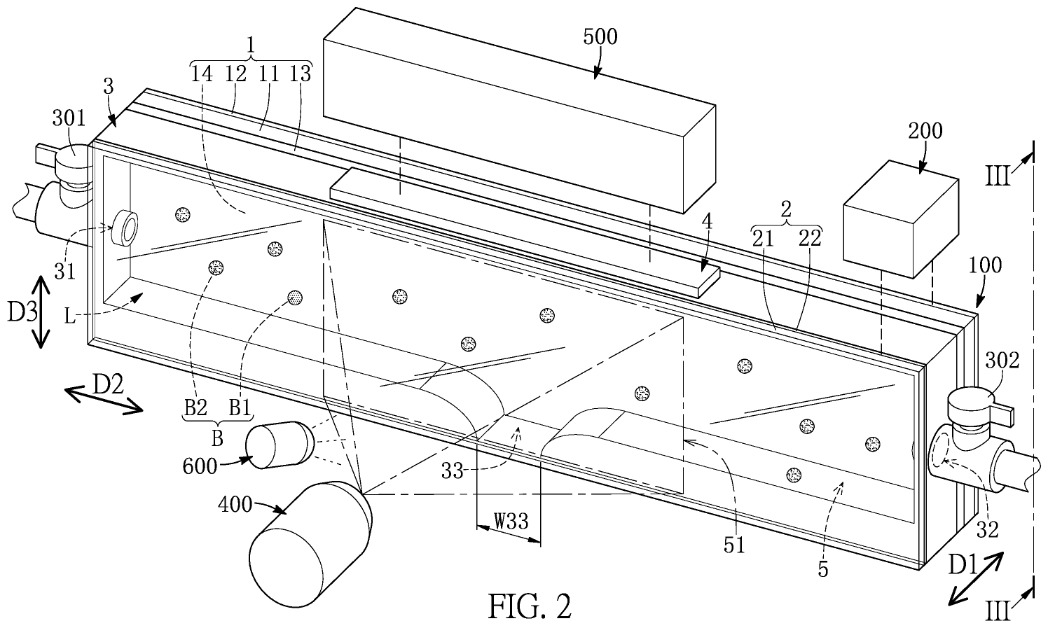

In addition, in order to enable the pico-droplet generator 100 to accurately form the pico-droplet L1 covering the at least one target bioparticle B1 for effectively implementing the selection and enrichment process of the at least one target bioparticle B1, the pico-droplet generator 100 is preferably used in cooperation with the light source mechanism 600. As shown in FIG. 2, FIG. 4, and FIG. 5, the light source mechanism 600 is arranged adjacent to the camera device 400, and the light source mechanism 600 is configured to move the at least one target bioparticle B1 into the pico-droplet emission region R by emitting light onto the at least one target bioparticle B1 to apply a dielectrophoretic (DEP) force F1 to the at least one target bioparticle B1 through the light sensing structure 1. Moreover, the light source mechanism 600 can be used to move the at least one non-target bioparticle B2 away from the pico-droplet emission region R by emitting light onto the at least one non-target bioparticle B2 to apply a DEP force F2 to the at least one non-target bioparticle B1 through the light sensing structure 1.

Accordingly, the pico-droplet generator 100 and the light source mechanism 600 of the bioparticle enrichment apparatus 1000 provided by the present embodiment can be used in cooperation with each other to allow the pico-droplet emission region R to quickly and accurately have the at least one target bioparticle B1 therein, such that the at least one target bioparticle B1 can be collected in the first container 700 by forming the pico-droplet L1.

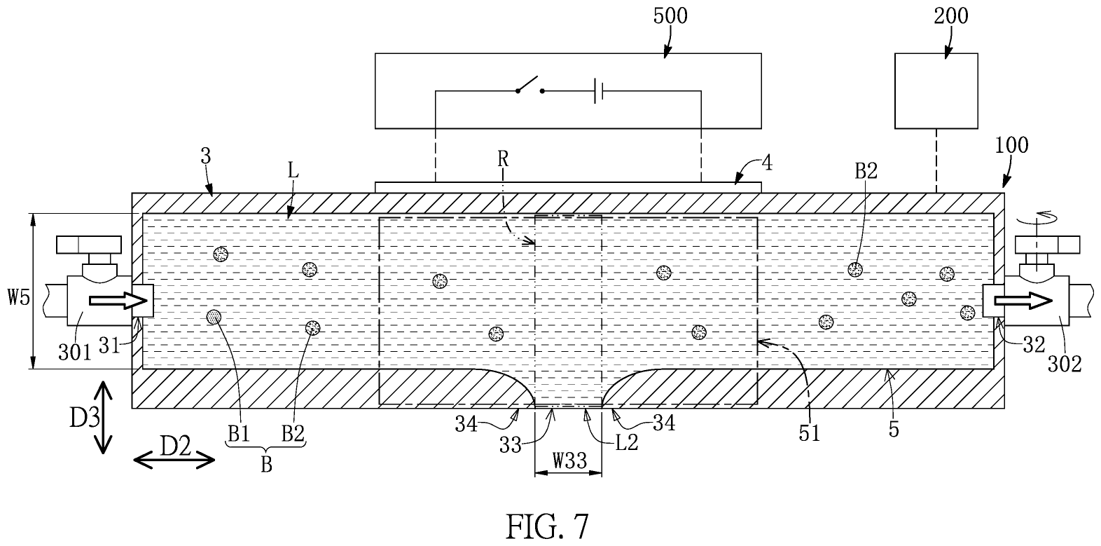

As shown in FIG. 1 and FIG. 7, after the at least one target bioparticle B1 located in the pico-droplet emission region R is collected in the first container 700, the second valve 302 can be opened again for allowing the liquid specimen L in the viewable segment 51 not having any target bioparticle B1 to flow into the second container 307, so that the non-target bioparticles B2 can be collected in the second container 307, but the present disclosure is not limited thereto.

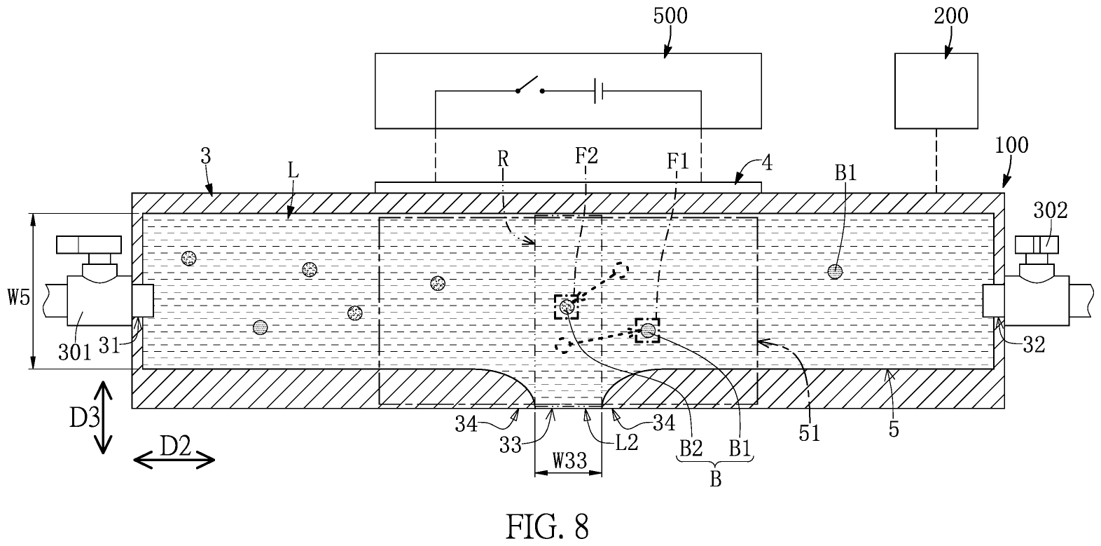

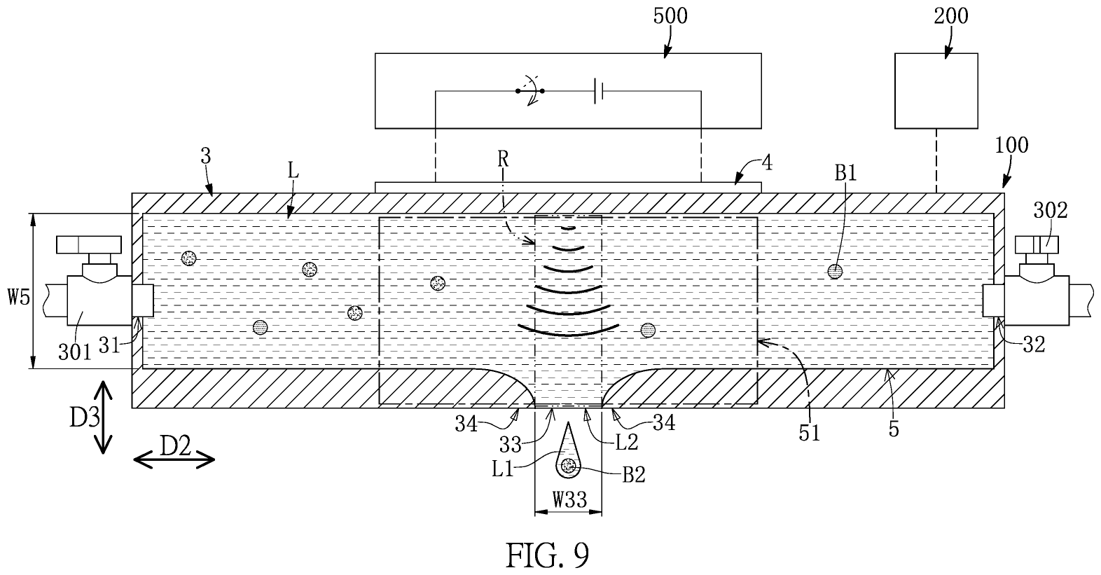

For example, as shown in FIG. 1, FIG. 8, and FIG. 9, the light source mechanism 600 can be configured to move the at least one target bioparticle B1 away from pico-droplet emission region R by emitting light onto the at least one target bioparticle B1 and can be configured to move the at least one non-target bioparticle B2 into the pico-droplet emission region R by emitting light onto the at least one non-target bioparticle B2, such that the at least one non-target bioparticle B2 is collected in the first container 700 by forming the pico-droplet L1, the at least one target bioparticle B1 is collected in the second container 307.

In addition, a quantity of the selection hole 33 of the pico-droplet generator 100 in the present embodiment is only one, but the present disclosure is not limited thereto. For example, in other embodiments of the present disclosure not shown in the drawings, the pico-droplet generator 100 can have a plurality of selection holes 33 spaced apart from each other and arranged along the flowing direction D2, and the pico-droplet generator 100 can further include a plurality of operation mechanisms (e.g., the piezoelectric members 4) respectively operated with the selection holes 33, so that the bioparticle enrichment apparatus 1000 can be used to select more than one kind of the bioparticles B according to different requirements through the selection holes 33 and the operation mechanisms.

### Second Embodiment

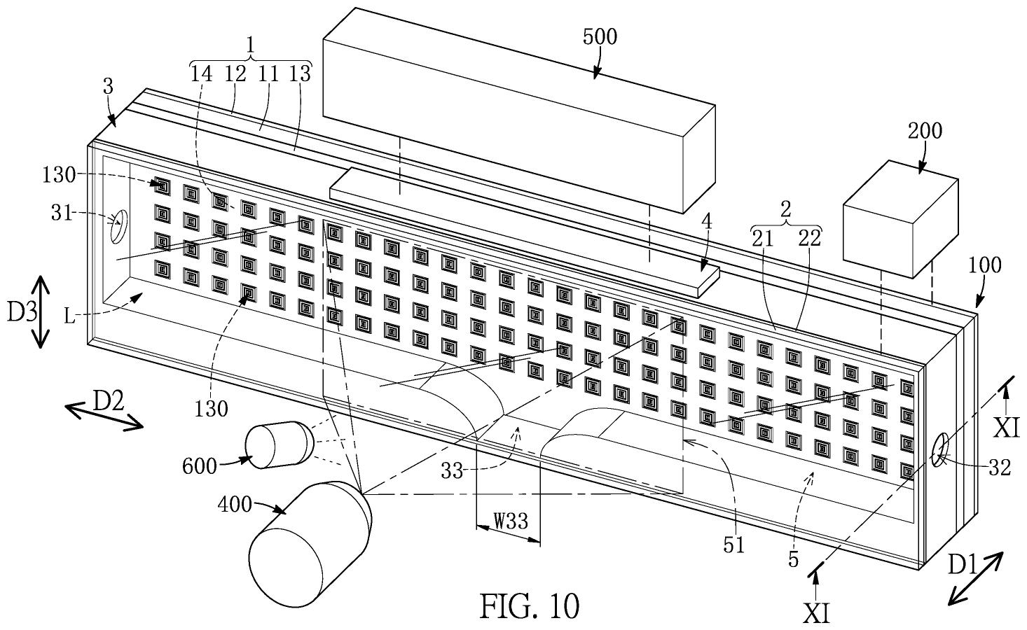

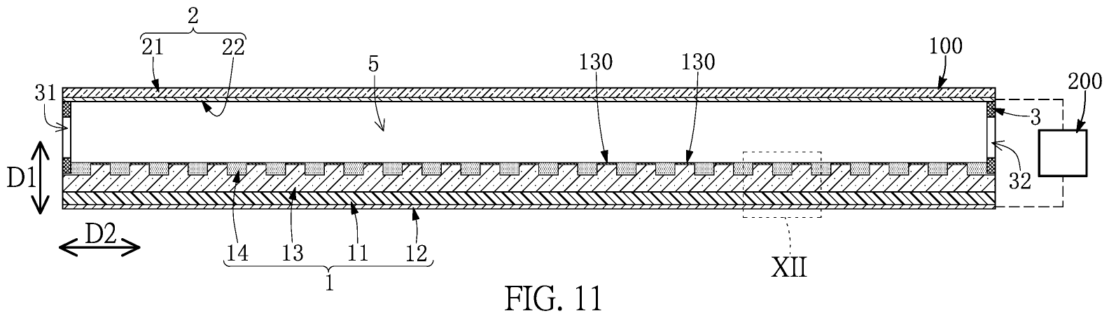

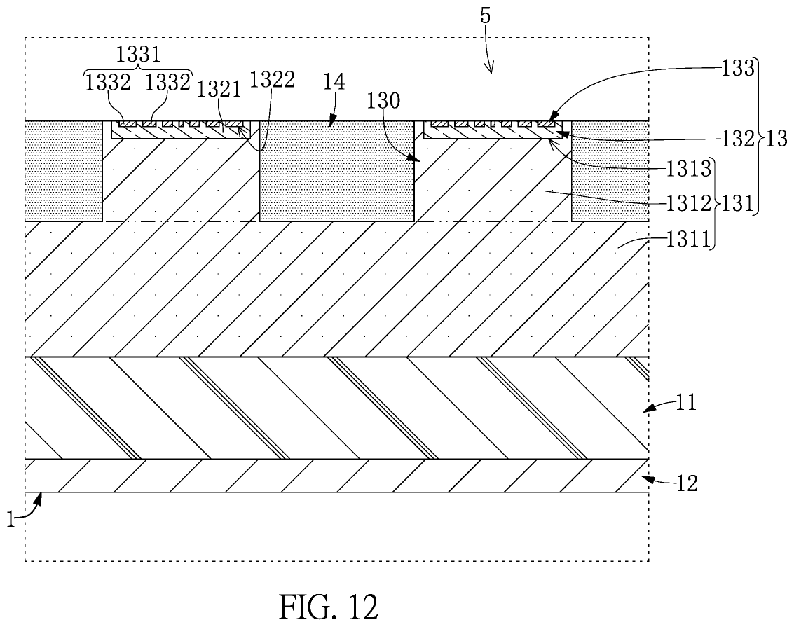

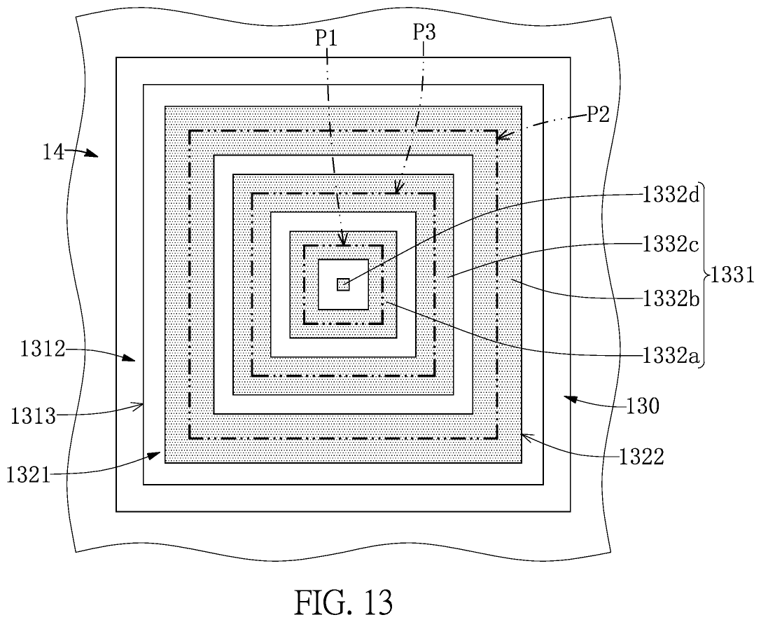

Referring to FIG. 10 to FIG. 13, a second embodiment of the present disclosure, which is similar to the first embodiment of the present disclosure, is provided. For the sake of brevity, descriptions of the same components in the first and second embodiments of the present disclosure will be omitted herein, and the following description only discloses different features between the first and second embodiments.

In the present embodiment, the photoelectric layer 13 can generate a better photoelectric coupling effect through a specific configuration. Specifically, the photoelectric layer 13 includes a collector layer 131 formed on the first substrate 11, a plurality of base regions 1321 formed in the collector layer 131, and a plurality of emitter regions 1331 that are respectively formed in the base regions 1321. In other words, the collector layer 131 is a N-type layer; the base regions 1321 are located at a same height with respect to the first electrode layer 12 and are jointly defined as a base layer 132 that is a P-type layer; and the emitter regions 1331 are located at a same height with respect to the first electrode layer 12 and are jointly defined as an emitter layer 133 that is a heavily doped N-type layer.

Specifically, the collector layer 131 in the present embodiment includes a connection layer 1311 formed on the first substrate 11 and a plurality of collector regions 1312 that are formed on the connection layer 1311 and that are spaced apart from each other. Moreover, an end (e.g., a top end) of each of the collector regions 1312 away from the first electrode layer 12 has a first slot-like portion 1313.

It should be noted that the collector regions 1312 of the present embodiment are electrically coupled to each other through the connection layer 1311, but the present disclosure is not limited thereto. For example, in other embodiments of the present disclosure not shown in the drawings, according to design requirements, the collector layer 131 can be provided without the connection layer 1311, the collector regions 1312 are directly formed on the first substrate 11, and the first substrate 11 can be a low-doped N-type layer, so that the first substrate 11 and the collector layer 131 can be jointly used as a collector.

The base regions 1321 are respectively formed in the first slot-like portions 1313 of the collector regions 1312 (i.e., each of the collector regions 1312 is formed with one of the base regions 1321 arranged therein), and an end (e.g., a top end) of each of the base regions 1321 away from the first electrode layer 12 has a plurality of second slot-like portions 1322 spaced apart from each other.

Moreover, the emitter regions 1331 are respectively formed in the base regions 1321 (i.e., each of the base regions 1321 is formed with one of the emitter regions 1331 arranged therein). Each of the emitter regions 1331 includes a plurality of emitter pads 1332 respectively formed in the second slot-like portions 1322 of a corresponding one of the base regions 1321.

In summary, each of the base regions 1321, a corresponding one of the collector regions 1312, and a corresponding one of the emitter regions 1331 are jointly formed as a vertical transistor 130. The insulating layer 14 in the present embodiment is a silicon nitride layer or a silicon oxide layer, but the present disclosure is not limited thereto. The insulating layer 14 covers the vertical transistors 130 and separates the vertical transistors 130 from each other, and an end (e.g., a top end) of each of the emitter pads 1322 away from the first electrode layer 12 is exposed from the insulating layer 14. In other words, the insulating layer 14 covers and is connected to the connection layer 1311 and a surrounding lateral surface of each of the vertical transistors 130.

As the vertical transistors 130 in the present embodiment are of substantially the same structure, the following description discloses the structure of just one of the vertical transistors 130 for the sake of brevity, but the present disclosure is not limited thereto. For example, in other embodiments of the present disclosure not shown in the drawings, the vertical transistors 130 can be of different structures.

In the present embodiment, the end of the collector region 1312, the end of the base region 1321, and the end of each of the emitter pads 1332 are preferably coplanar with each other, each of the emitter pads 1332 has a width greater than a thickness thereof, and any two of the emitter pads 1332 adjacent to each other have a distance therebetween that is less than 5 μm, but the present disclosure is not limited thereto. In addition, a thickness of the base region 1321 is within a range from 15% to 35% of a thickness of the collector region 1312, and the thickness of each of the emitter pads 1332 is within a range from 5% to 20% of the thickness of the base region 1321, but the present disclosure is not limited thereto.

It should be noted that from the perspective of the bioparticles, any slight change in the pico-droplet generator 100 would have a significant influence thereon. Accordingly, the above description in the present embodiment describes the size and arrangement of the emitter pads 1332 of the vertical transistor 130 that are provided to facilitate the selection of the bioparticles by an electric field difference that is progressively distributed, but the present disclosure is not limited thereto.

In the vertical transistor 130 of the present embodiment, the emitter pads 1332 include a first pad 1332a arranged along an inner ring-shaped path P1, a centric pad 1332d arranged inside of the inner ring-shaped path P1, a second pad 1332b arranged along an outer ring-shaped path P2, and a third pad 1332c that is arranged along an additional ring-shaped path P3. The outer ring-shaped path P2 surrounds the inner ring-shaped path P1, and the additional ring-shaped path P3 is located between the inner ring-shaped path P1 and the outer ring-shaped path P2, but the present disclosure is not limited thereto. For example, in other embodiments of the present disclosure not shown in the drawings, the centric pad 1332d and/or the third pad 1332c can be omitted according to design requirements; or, a quantity of the additional ring-shaped path P3 between the inner ring-shaped path P1 and the outer ring-shaped path P2 can be at least two.

Specifically, a width of the centric pad 1332d, a width of the first pad 1332a, and a width of the second pad 1332b are different from each other, and a width of the third pad 1332c is within a range from the width of the first pad 1332a to the width of the second pad 1332b. In the present embodiment, widths of the emitter pads 1332 gradually decrease in a direction from the outer ring-shaped path P2 toward the centric pad 1332d. In other words, the emitter pads 1332 can be listed as follows in an order from largest to smallest in width: the second pad 1332b, the third pad 1332c, the first pad 1332a, and the centric pad 1332d, but the present disclosure is not limited thereto.

In addition, the specific distribution, quantity, and shape of the first pad 1332a, the second pad 1332b, and the third pad 1332c can be adjusted or changed according to design requirements, but the present disclosure is not limited thereto.

In summary, any one of the vertical transistors 130 of the pico-droplet generator 100 is configured to be irradiated by the light source mechanism 600 so as to allow a plurality of DEP forces to be applied to move the bioparticles (e.g., the at least one target bioparticle) through a distribution of the emitter pads 1332 and an electric field difference that is generated in the liquid specimen from non-uniform electric fields of the emitter pads 1332.

Accordingly, the photoelectric layer 13 of the bioparticle enrichment apparatus 1000 provided by the present embodiment has a specific structural design, so that the emitter pads 1332 of any one of the vertical phototransistors 130 can be used in a contactless photoelectric coupling manner to generate the electric fields jointly forming the electric field difference that is similar to a corona discharge, thereby enabling the emitter pads 1332 of any one of the vertical phototransistors 130 to accurately move (or capture) the at least one target bioparticle to any position.

### Beneficial Effects of the Embodiments

In conclusion, the configuration of the pico-droplet generator of the bioparticle enrichment apparatus provided by the present disclosure can be used to quickly and accurately implement a selection and enrichment process of the bioparticle (or the at least one target bioparticle) through the piezoelectric member. The pico-droplet generator in the present disclosure has an architecture that allows for mass-production of the same, thereby facilitating reduction of the overall cost of the bioparticle enrichment apparatus.

Moreover, the pico-droplet generator and the light source mechanism of the bioparticle enrichment apparatus provided by the present disclosure can be used in cooperation with each other to allow the pico-droplet emission region to quickly and accurately have the at least one target bioparticle therein, such that the at least one target bioparticle can be collected in the first container by forming the pico-droplet.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A bioparticle enrichment apparatus provided for selecting at least one of bioparticles from a liquid specimen having the bioparticles, the bioparticle enrichment apparatus comprising:
a pico-droplet generator including:
a light sensing structure including:
a first substrate;
a first electrode layer and a photoelectric layer respectively formed on two opposite sides of the first substrate; and
an insulating layer covering the photoelectric layer;
a mating structure spaced apart from the light sensing structure, wherein at least one of the mating structure and the light sensing structure is transparent, and the mating structure includes a second substrate that faces toward the light sensing structure and a second electrode layer that is formed on the second substrate;
a bonding layer that is connected in-between the light sensing structure and the mating structure along a thickness direction so as to jointly define a selection channel along a flowing direction perpendicular to the thickness direction, wherein at least one of the mating structure and the bonding layer has an inlet that is located at an upstream of the selection channel and an outlet that is located at a downstream of the selection channel, and wherein the bonding layer has a selection hole that is in spatial communication with the selection channel along a dripping direction perpendicular to the thickness direction and the flowing direction; and
a piezoelectric member disposed on the bonding layer, wherein the piezoelectric member and the selection hole are respectively located at two opposite sides of the bonding layer along the dripping direction, and wherein the pico-droplet generator has a pico-droplet emission region defined as extending from the selection hole toward the piezoelectric member;
a power device electrically coupled to the first electrode layer and the second electrode layer;
a pressure balance device including a first valve that is assembled to the inlet and a second valve that is assembled to the outlet, wherein the pressure balance device is configured to control a velocity and a pressure of the liquid specimen in the selection channel through the first valve and the second valve;
a camera device corresponding in position to a viewable segment of the selection channel, wherein the pico-droplet emission region is arranged in the viewable segment, and the camera device is configured to take a real-time image of the liquid specimen in the viewable segment; and
a control device electrically coupled to the piezoelectric member, wherein, when the real-time image obtained by the camera device shows that at least one of the bioparticles is located in the pico-droplet emission region, the control device allows the piezoelectric member to output a pico-droplet by driving the liquid specimen in the pico-droplet emission region to pass through the selection hole, and wherein the pico-droplet covers the at least one of the bioparticles.

2. The bioparticle enrichment apparatus according to claim 1, wherein the bioparticles located in the viewable segment have at least one target bioparticle, and the bioparticle enrichment apparatus further includes:
a light source mechanism that is configured to move the at least one target bioparticle into the pico-droplet emission region by emitting light onto the at least one target bioparticle to apply a dielectrophoretic (DEP) force to the at least one target bioparticle through the light sensing structure.

3. The bioparticle enrichment apparatus according to claim 2, wherein the bioparticles located in the viewable segment has at least one non-target bioparticle, and the light source mechanism is configured to move the at least one non-target bioparticle away from the pico-droplet emission region by emitting light onto the at least one non-target bioparticle to apply a DEP force to the at least one non-target bioparticle through the light sensing structure.

4. The bioparticle enrichment apparatus according to claim 2, wherein the photoelectric layer includes:
a collector layer formed on the first substrate, wherein the collector layer includes a plurality of collector regions spaced apart from each other, and an end of each of the collector regions away from the first electrode layer has a first slot-like portion;
a plurality of base regions respectively formed in the first slot-like portions of the collector regions, wherein an end of each of the base regions away from the first electrode layer has a plurality of second slot-like portions spaced apart from each other; and
a plurality of emitter regions respectively formed in the base regions, wherein each of the emitter regions includes a plurality of emitter pads respectively formed in the second slot-like portions of a corresponding one of the base regions, wherein each of the base regions, a corresponding one of the collector regions, and a corresponding one of the emitter regions are jointly formed as a vertical transistor, and wherein the insulating layer covers the vertical transistors and separates the vertical transistors from each other, and an end of each of the emitter pads away from the first electrode layer is exposed from the insulating layer;
wherein any one of the vertical transistors is configured to be irradiated by the light source mechanism so as to enable a plurality of DEP forces to be applied to move the at least one target bioparticle through a distribution of the emitter pads and an electric field difference that is generated from non-uniform electric fields of the emitter pads.

5. The bioparticle enrichment apparatus according to claim 4, wherein, in each of the vertical transistors, the emitter pads include a first pad arranged along an inner ring-shaped path and a second pad that is arranged along an outer ring-shaped path surrounding the inner ring-shaped path, and a width of the first pad is different from a width of the second pad.

6. The bioparticle enrichment apparatus according to claim 5, wherein, in each of the vertical transistors, the emitter pads include a third pad arranged along an additional ring-shaped path located between the inner ring-shaped path and the outer ring-shaped path, and a width of the third pad is within a range from the width of the first pad and the width of the second pad.

7. The bioparticle enrichment apparatus according to claim 2, wherein, when the piezoelectric member or the light source mechanism is in operation, the pressure balance device maintains the pressure of the liquid specimen in the selection channel through the first valve in an open mode and the second valve in a closed mode.

8. The bioparticle enrichment apparatus according to claim 1, wherein the pico-droplet generator has a hydrophobic surface surrounding the selection hole, and the pressure balance device enables the liquid specimen to form a liquid level in the selection hole that is coplanar with an outer surface of the bonding layer.

9. The bioparticle enrichment apparatus according to claim 8, further comprising a first container for receiving the pico-droplet, wherein the pressure balance device includes:
an air pump;
a switch connected to the air pump;
a pressure balance bottle being in fluid communication with the air pump and the switch;
a liquid injection bottle being in fluid communication with the switch and the first valve, wherein the liquid injection bottle enables the liquid specimen received therein to be injected into the selection channel through the first valve and the inlet; and
a second container being in fluid communication with the second valve.

10. The bioparticle enrichment apparatus according to claim 1, wherein a width of the selection hole along the flowing direction is within a range from 40 μm to 300 μm, and a boundary of the viewable segment is spaced apart from a center of the selection hole along the flowing direction by a distance within a range from 100 μm to 300 μm.

11. The bioparticle enrichment apparatus according to claim 1, wherein the pico-droplet emission region is arranged in a projection area defined by orthogonally projecting the selection hole onto the piezoelectric member along the dripping direction.

12. The bioparticle enrichment apparatus according to claim 11, wherein a boundary of the pico-droplet emission region is spaced apart from a center of the selection hole along the dripping direction by a distance within a range from 50 μm to 200 μm.

13. A bioparticle enrichment device provided for selecting a target bioparticle from a liquid specimen having bioparticles, the bioparticle enrichment device comprising:
a pico-droplet generator including:
a light sensing structure including:
a first substrate;
a first electrode layer and a photoelectric layer respectively formed on two opposite sides of the first substrate; and
an insulating layer covering the photoelectric layer;
a mating structure spaced apart from the light sensing structure, wherein at least one of the mating structure and the light sensing structure is transparent, and the mating structure includes a second substrate that faces toward the light sensing structure and a second electrode layer that is formed on the second substrate;
a bonding layer that is connected in-between the light sensing structure and the mating structure along a thickness direction so as to jointly define a selection channel along a flowing direction perpendicular to the thickness direction, wherein at least one of the mating structure and the bonding layer has an inlet that is located at an upstream of the selection channel and an outlet that is located at a downstream of the selection channel, and wherein the bonding layer has a selection hole that is in spatial communication with the selection channel along a dripping direction perpendicular to the thickness direction and the flowing direction; and
a piezoelectric member disposed on the bonding layer, wherein the piezoelectric member and the selection hole are respectively located at two opposite sides of the bonding layer along the dripping direction, and wherein the pico-droplet generator has a pico-droplet emission region defined as extending from the selection hole toward the piezoelectric member; and
a light source mechanism that is configured to move the target bioparticle into the pico-droplet emission region by emitting light onto the target bioparticle to apply a dielectrophoretic (DEP) force to the target bioparticle through the light sensing structure, wherein the piezoelectric member is configured to output a pico-droplet by driving the liquid specimen in the pico-droplet emission region to pass through the selection hole, and wherein the pico-droplet covers the target bioparticle.

14. A pico-droplet generator, comprising:
a light sensing structure including:
a first substrate;
a first electrode layer and a photoelectric layer respectively formed on two opposite sides of the first substrate; and
an insulating layer covering the photoelectric layer;
a mating structure spaced apart from the light sensing structure, wherein at least one of the mating structure and the light sensing structure is transparent, and the mating structure includes a second substrate that faces toward the light sensing structure and a second electrode layer that is formed on the second substrate;
a bonding layer that is connected in-between the light sensing structure and the mating structure along a thickness direction so as to jointly define a selection channel along a flowing direction perpendicular to the thickness direction, wherein at least one of the mating structure and the bonding layer has an inlet that is located at an upstream of the selection channel and an outlet that is located at a downstream of the selection channel, and wherein the bonding layer has a selection hole that is in spatial communication with the selection channel along a dripping direction perpendicular to the thickness direction and the flowing direction; and
a piezoelectric member disposed on the bonding layer, wherein the piezoelectric member and the selection hole are respectively located at two opposite sides of the bonding layer along the dripping direction, and wherein the pico-droplet generator has a pico-droplet emission region defined as extending from the selection hole toward the piezoelectric member.

15. The pico-droplet generator according to claim 14, wherein the pico-droplet generator has a hydrophobic surface surrounding the selection hole, a width of the selection hole along the flowing direction is within a range from 40 μm to 300 μm, the pico-droplet emission region is arranged in a projection area defined by orthogonally projecting the selection hole onto the piezoelectric member along the dripping direction, and a boundary of the pico-droplet emission region is spaced apart from a center of the selection hole along the dripping direction by a distance within a range from 50 μm to 200 μm.
